(19) United States
(12) Patent Application Publication
(10) Pub. No.: US 2023/0284633 A1
(43) Pub. Date: Sep. 14, 2023

(54) CARRABIITOL FORMULATION TO MAINTAIN OSMOTIC BALANCE IN PLANTS AGAINST ABIOTIC STRESS AND METHOD OF EXTRACTION & PREPARATION THEREOF

(71) Applicant: M/S. PUSHPA J. SHAH, Gujarat (IN)

(72) Inventors: Neil J. Shah, Gujarat (IN); Femida Y. Patel, Gujarat (IN)

(21) Appl. No.: 18/040,048

(22) PCT Filed: Sep. 24, 2021
(86) PCT No.: PCT/IN2021/050935

§ 371 Date: Jan. 31, 2023

(30) Foreign Application Priority Data
Sep. 24, 2020 (IN) 202021041518

(51) Int. Cl. A01N 65/03; A01N 43/16; A01P 21/00

(52) U.S. Cl. CPC A01N 65/03; A01N 43/16; A01P 21/00

(57) ABSTRACT

The present invention provides seaweed based carrabiitol® formulation for maintaining osmotic balance in plants against abiotic stress and method for obtaining the same. The formulation comprises at least one carrageenan 5 oligosaccharide derivative polyol having osmo-protectant activity inducing abiotic stress tolerance, increase adaptation of plants to drastically changing environmental conditions, resulting in better growth and yield.

### RELATED APPLICATION

This application bears priority from Provisional Application No: 202021041518 dated 24th September 2020 and bearing the Title: “Carrabiitol formulation to maintain osmotic balance in plants against abiotic stress and method of extraction & preparation thereof”.

### TECHNICAL FIELD

The present invention relates to a carrabiitol formulation based on seaweed derived oligosaccharide polyols, its method of extraction and application for inducing abiotic stress tolerance in plants, leading to enhanced plant growth and vigour.

### BACKGROUND

Plants being exposed to different environmental stresses learn and adopt to these stresses in number of ways. The term ‘abiotic stress’ includes numerous stresses caused by complex environmental conditions, e.g. strong light, UV, high and low temperatures, freezing, drought, salinity, heavy metals and hypoxia. These stresses will increase in the near future because of global climate change, according to reports from the Intergovernmental Panel of Climate Change. In India as well as certain parts of Asia almost 70-80% of farming is dependent on monsoons and due to inconsistency in its onset (delayed or early monsoon) the crops are exposed to drought or water stress. The plants exposed to such stress that may last from a week to two weeks’ time, may display reduced vigour, reduced plant growth as well as reduction in productivity and yield. Similarly during winters the crops are usually exposed to short periods of frost leading to abiotic stress that affects plants yield and productivity. It is during such conditions of stress, interventions are required that will mitigate the effect of abiotic stress helping the crops to overcome the short but detrimental stress conditions. Plant physiological development and productivity is largely affected by many environmental stresses such as drought, high salinity, and extreme temperatures variations etc. These stresses trigger expression of an array of gene in response. Thus, the expressed products of these genes function not only in stress response but also in stress tolerance. In view of this many abiotic stress-inducible genes have been isolated and their functions precisely characterized in transgenic plants. The availability of these research data and analysis have provided varied insights into abiotic stress responses and tolerance in plants. Advancements in gene editing and manipulation will prove valuable for super crops using super genes from different sources by improving their ability to accumulate osmolytes for better productivity as well as ensure survival under various abiotic stresses.

However, the development of transgenic plant is high cost and vital process. A necessary and effective alternative could be to develop formulations based on various osmolytes such as - amino acids (e.g. proline), polyamines and quaternary amines (e.g. glycinebetaine), polyol (e.g. mannitol, trehalose) and sugars like sucrose and oligosaccharides. Such formulations can provide cost effective adoptable and environment-friendly solution to the problem of abiotic stress.

Another approach to enhance the abiotic stress tolerance of plants is the identification of sustainable agri-inputs which are able to confer abiotic stress tolerance on plants. However, more emphasis on crop hybrids and genetically modified crops leads to focus on monocultures and the use of heirloom varieties eventually becomes a thing of the past. Thus, it adds to the now emerging problem of loss of natural/wild type varieties of the concerned plant species.

Osmolytes play pivotal role in plants for improving abiotic stress tolerance. The over-production and accumulation of osmo-protectants is universal process for combating abiotic stress. In several studies, the biosynthesis and regulatory genes from different sources have been successfully utilized for generating abiotic stress-tolerant transgenic plants which have better capability to rapidly accumulate sufficient amounts of osmolytes. From various reports, it is clear that the different candidates for the osmolyte biosynthesis pathway can be utilized for better survival of crop plants. In the era of exploding populations and shrinking agricultural land, we need to focus on utilizing the benefits of osmolyte-mediated crop improvement.

Osmotic adjustment (OA) is referred as a net increase in solute concentration and is perceived as important survival mechanism to drought stress. As water is being removed from the plant cells, its osmotic potential is reduced due to the effect of solute concentration. However, during the course of cellular water loss, solutes are actively accumulated. This reduces the out flow of water from cell, thereby reducing loss of turgor and allows stomatal opening and expansion growth to continue progressively at lower water potentials.

Osmolytes are low molecular weight and soluble compounds. Many studies indicated that the accumulation of compatible solutes in plants causes resistance to various stresses such as drought, high temperature and high salinity. The primary function of compatible solutes is to prevent water loss to maintain cell turgor and to maintain the gradient for water uptake into the cell, protect and stabilize 3D structure of proteins and photosynthetic apparatus (Papageorgiou & Murata, 1995. Photosynthesis Research 44,243-252, 1995); regulate cellular osmotic adjustment and detoxify reactive oxygen stresses. Upon relief from stress, these solutes are metabolized and are considered as an important energy source (Hare PD, Van Staden J. 1998. Plant Cell and Environment 21: 535-553). Compatible solutes are divided into three major groups - amino acids (e.g. proline), polyamins and quaternary amines (e.g. glycinebetaine), polyol (e.g. mannitol, trehalose) and sugars like sucrose and oligosaccharides (Hare PD, Van Staden J. 1998. Plant Cell and Environment 21: 535-553). Seed treatment with osmolytes and plant growth regulating chemicals can help the seeds to overcome water stress.

In literature, it is well proven that seaweed derived oligosaccharide with different process gives structurally different oligosaccharide with different activities. Researchers are working on the various combinations of chemicals to produce most effective oligosaccharide and its derivative oligosaccharide alditol to combat antiviral effect and antioxidant activity in various fields. For different fields detailed study and research is required. In agriculture, researchers are focusing on whole utilization of seaweeds and combination of natural actives like humic acid, plant extracts and other beneficial bio-stimulant (US6893479). US6893479 patent claims a growth promoting composition comprising seaweed sap that has growth promoting substances and micronutrients.

The use of these bio-stimulants leads to plant’s better growth; however, in extreme stress conditions, the effectivity of those bio-stimulant is questionable.

Currently seaweed extracts (SE) are widely used as plant biostimulants, which are ‘any substance or microorganism applied to plants with the aim to enhance nutrition efficiency, abiotic stress tolerance and/or crop quality traits, regardless of its nutrients content (Du Jardin 2015, P. Sci. Hortic. Vol196, pp. 3-14). Seaweeds extracts biochemical composition is complex (polysaccharides, minerals, vitamins, oils, fats, acids, antioxidants, pigments, hormones) (Craigie et al. 2011: J. Appl. Phycol. Vol 23, pp 371-393; Khan et al. 2009: Plant Growth Regul. Vol 28, pp 386-399; and Michalak & Chojnacka 2014: Eng. Life Sci. Vol 14, pp 581-591). Hence understanding their mechanism of action is highly intricate, and often requires multidisciplinary approach due the multiple interaction between the substantial numbers of bioactive compound within the same extract. The synergistic effect of these compounds adversely effect the plant cell integrity and unfold protein structure in extreme abiotic stress condition due to triggering of multiple signalling pathway which leads to accumulation of various solutes into the plant, which create osmotic stress to cell . Trivedi et al. investigated the effect of a seaweed extract based on Kappaphycus alvarezii on alleviating water deficit stress in maize and concluded that even if the level of some antioxidants was enhanced like APX, the yield was not improved significantly (Trivedi et al 2018: Algal Res. Vol: 35, 236-244). Hence, it is requirement of developing formulation for extreme abiotic stress condition which contain osmolyte which trigger specific pathway for specific abiotic stress tolerance.

To address the above issue of plant abiotic stress, there is a need to develop or design technology that overcome the problems associated with prior arts. The present invention provides a simplified, pH agnostic, stable seaweed based carrageenan oligosaccharide polyols formulation (carrabiitol® formulation) that is free of bioactives and has osmo-protectant properties. Unlike other commercially available extracts or biostimulants, that may have mixture of untargeted molecules, the formulation of the present invention has only carrageenan oligosaccharides derivative polyols, is completely free of bioactives and have a minimal presence of heavy metals. This helps remove all possible potential interference and activates a precise plant signalling pathway under abiotic stress conditions thereby clearly establishing a positive effect on crop yields. Upon relief from stress, carrageenan oligosaccharides derivative polyols are metabolized and become an important energy source for plant metabolic pathways.

### SUMMARY OF THE INVENTION

One of the aspects of the present invention is to provide a carrabiitol® formulation for maintaining osmotic balance in plants against abiotic stress, the formulation comprising at least one carrageenan oligosaccharide derivative polyol, wherein the carrageenan oligosaccharide derivative polyol is selected from a group of di-saccharide polyols, trisaccharide polyols, tetra-saccharides polyols, hexa-saccharide polyols and possible derivatives at most up to about 60%, expressed in percent relative to the total carrageenan oligosaccharide derivative polyol and at least one carrageenan oligosaccharide derivative polyol has molecular weight in the range of 200 to 12,000 dalton.

Another aspect of the present invention is to provide a method for obtaining carrabiitol® formulation comprising at least one carrageenan oligosaccharide derivatives polyol from seaweed(s), the method comprising:
a) processing the seaweed(s) to obtain first residue and first filtrate;
b) diluting the first residue obtained from step (a) with water upto 35% and adjusting the pH in the range of 1 to 5.5 with organic acid and heating at a temperature in the range of 50 to 150° C. for the time period in the range of 30 minutes to 300 minutes to obtain a solution;
c) cooling the solution to ambient temperature and centrifuging/filtering to obtain second residue and second filtrate;
d) treating the second filtrate with a metallic ion complex at a temperature in the range of 30° C. to 110° C. for a time period in the range of 30 minute to 300 minutes under continuous stirring followed by neutralising with an alkali solution to obtain a neutralised solution;
e) adding a suitable preservative to the neutralised solution to obtain the carrageenan oligosaccharide derivative polyol; wherein the carrageenan oligosaccharide derivative polyol thus obtained has osmo-protectant activity; and
f) evaporating, drying and sieving the carrageenan oligosaccharide derivative polyol to obtain the carrabiitol® formulation.

### DESCRIPTION OF ACCOMPANYING GRAPHS AND FIGURES

Graph 1(a, b, c) represents that in sorghum concentration upto 100 mM seeds are capable to overcome stress at large extent at their own but where concentration of salts increased, the capacity of overcoming stress decreases. If seeds are coated with carrabittol formulation, results are significantly increased by 33.32% (see control values). Thus, indicating that formulation is effective even at higher concentration of salts to overcome stress.

Graph 2 (a, b, c) shows comparison of Stress Tolerance index %.

Graph 3 (a, b, c) represents root length of Sorghum in Abiotic Stress Condition. Graph represent that in stress condition, root length is too low from 0.73 cm to 5.17 cm (Control), whereas in formulation root length observed is 6.30 to 11.47 cm. It shows that formulation also stimulate root growth along with germination percentage.

Graph 4 (a, b, c) illustrates shoot length of Sorghum in Abiotic Stress Condition.

FIG. 1 depicts comparison of Root and Shoot length in Excess Water stress Condition in Sorghum.

FIG. 2 shows Sorghum Growth in drought stress (a) 3000 ppm (Stressed seeds and with coat of Carrabiitol Formulation) (b) Control (only Stressed seeds without Formulation)

FIG. 3 shows Sorghum Growth in Salinity Stress Condition (a) 3000 ppm (b) Control

FIG. 4 represents HPLC-RI Profiling of Carrabiitol formulation

FIG. 5 shows (a) 1H NMR Spectrum, b) 13 C NMR Spectrum

FIG. 6 shows LC-MS (ESI-MS) of Carrageenan oligosaccharide derivative polyols (A) Dimer, (B) Trimer, (C) Tetramer, (D) Hexamer

FIG. 7 shows represents HPLC chromatogram of Carrageenan oligosaccharide mixture and Carrabiitol; (a)Carrageenan oligosaccharide mixture before stability (b) Carrageenan oligosaccharide mixture after stability, (c) Carrabiitol before stability, and (d) Carrabiitol after stability

FIG. 8 shows wheat kernel of control and Carrabiitol formulation

FIG. 9 illustrates phytotoxic experiment of Carrabiitol formulation (1) at 1% concentration, (2) 5% Concentration

### DETAILED DESCRIPTION OF THE INVENTION

The present disclosure is subject to variations and modifications other than those specifically described. Those skilled in the art would understand that the present disclosure includes all such variations and modifications, including different steps, features, compositions and compounds referred to or indicated in this specification, individually or collectively, and any and all combinations of any or more of such steps or features. The present application describes certain materials and methods to exemplify the invention, however, any methods and materials similar or equivalent variations of those described herein can be used in the practice or testing of the disclosure.

Further, unless defined otherwise, all technical and scientific terms used herein have the same connotation as commonly understood by a person having ordinary skill in the art.

Where the term “comprising”, “comprises” is used in the present description, it does not exclude other elements or steps.

Where an indefinite or definite article is used when referring to a singular noun e.g. “a” or “an”, “the”, this includes a plural of that noun unless something else is specifically stated.

The present disclosure uses certain terms to describe the invention. Definition of those terms is as provided below:

### Definitions

The term “formulation” or “composition” as used herein refers to a seaweed derived product, used individually or in combination with other components for increasing plant growth and reducing plant abiotic stress, leading to enhanced plant growth and plant health.

The terms “formulation” and “composition” have been used interchangeably in this application.

The term “at least one” used herein means one or more and thus includes individual components as well as mixtures/combinations.

The said formulation is named Carrabiitol® and has been registered under Class 1 (Trade Mark No. 4514864) at the Trademark Registry, Mumbai. The said product is herein also referred simply as carrabiitol or carrabiitol granules or carrabiitol formulation or composition.

The term “adjuvant” used herein refers to an ingredient that improves the action of the principal ingredient. The adjuvant used in the present invention includes but not limited to surfactants, wetting agent, oils, thickening agent, stickers, spreaders, foaming agents, humectants, UV absorbants, emulsifiers, dispersants, stabilizing agent, coupling agent, cosolvents, compatibility agent, buffering agent, antifoam agent and anti-freezing agent.

The term “plant growth supporting substrate” used herein refers to the surface on which an organism (such as a plant, fungus) lives. They are referred to as soils, root media, growing media, root substrates or substrates, the materials that are placed into containers and in which the plants’ roots develop can have a profound effect on the development of the crop. This is because, regardless of its composition, the substrate may fulfil up to five basic functions: provide physical support for the plant; retain water in a form available to the plant; provide for gas exchange between the roots and the atmosphere outside of the container; serve as a reservoir for plant nutrients; sustain a microorganism population important in nutrient cycles and disease suppression.

The term “seaweed/s” refers to sea algae including and not limited to green, brown and red algae.

The term “bio-actives” used herein refers to any type of chemical compound synthesized or released by living organisms which has activity to improve the overall health of living organisms. For example, phytohormones, vitamins, amino acids, betaines etc.

The terms “extraction” or “production” or “obtaining” used herein refers to a seaweed derived component and the process employed to get the said product is referred as production/extraction/obtaining which is achieved by using the process described herein. The terms “extraction”, “production” and “obtaining” are used interchangeably in the specification.

The term “microencapsulation” used herein refers to a process of encapsulating microbes with a diameter of 1-1000 µm.

The term “encapsulation” used herein means inclusion of one thing within another thing so that the included thing is not apparent. In the present invention encapsulation of fertilizers, minerals, and agricultural supplements is carried out to increase the efficacy of said formulation required for plant growth.

The term “plant species” used herein includes agricultural, silvicultural, ornamental and horticultural plants that can be harvested or cultivated on a commercial scale or which serve as an important source of feed, food, fibres, fuel or sources of fuels or sources of other chemical compounds. Such plant species can be natural or wild types or modified by conventional or molecular breeding methods, mutagenesis or genetically modified.

The term “planting material” used herein refers to type of material used for the establishment of fields or parts of plants used for vegetative reproduction. Generally, planting material falls into two types of categories.
1. Conventional Planting material - For example, seeds, fruits, aggregates fruits, parts of aggregates fruits, bulbs, tubers, Suckers and bits and
2. Tissue culture plantlets - Plantlets produced by tissue culture under sterile medium using apical meristem of root and shoot of plants.

The term “agri-inputs” used herein refers to any external source which improves yield when applied to plant or soils, for example, fertilizers, organic fertilizer, biofertilizers, plant growth promoting substances and agrochemicals.

The term “possible derivatives” used herein refers to derivatives of carrageenan oligosaccharide polyols like carrageenan penta oligosaccharide polyol, hepta- oligosaccharide polyol and octa-oligosaccharide polyol when subject to hydrolytic reduction.

The term “osmoprotectant activity” used herein refers ability of compounds/ molecules that act as osmolytes and help organisms (plants) to survive extreme osmotic stress.

The term “metal ion complex” used herein refers to a metal ion that is bonded via coordinate covalent bonds to a small number of anions or neutral molecules called ligands. The metal ion complex used as reducing agent.

The term “reducing agent’ used herein refers an agent that remove oxygen from another substance or add hydrogen to it. The term “metal ion complex” or “reducing agent’ can be used interchangeably in the specification.

The present invention is exemplified in the application through embodiments, and do not limit the scope of the invention. All functionally equivalent components and products; compositions and formulations, and processes/methods are clearly within the scope of the disclosure, as described herein.

The present disclosure recites a formulation composition comprising seaweed derived oligosaccharide polyols, which promote plant resistance to abiotic stress caused by drought, salinity, extreme temperatures and water stress among others.

One of the embodiments of the present invention provides a carrabiitol® formulation for maintaining osmotic balance in plants against abiotic stress, the formulation comprising: at least one carrageenan oligosaccharide derivative polyol, wherein the carrageenan oligosaccharide derivative polyol is selected from a group of di-saccharide polyols, trisaccharide polyols, tetra-saccharides polyols, hexa-saccharide polyols and possible derivatives at most up to about 60%, expressed in percent relative to the total carrageenan oligosaccharide derivative polyol.

Another embodiment of the present invention provides a carrabiitol® formulation for maintaining osmotic balance in plants against abiotic stress, wherein at least one carrageenan oligosaccharide derivative polyol has molecular weight in the range of 200 - 12,000 dalton.

Another embodiment of the present invention provides a carrabiitol® formulation for maintaining osmotic balance in plants against abiotic stress, wherein said formulation further comprises at least one adjuvant ranging from 0.1% to 10%.

Yet another embodiment of the present invention provides a carrabiitol® formulation for maintaining osmotic balance in plants against abiotic stress, wherein at least one adjuvant is selected from surfactants, wetting agent, oils, thickening agent, stickers, spreaders, foaming agents, humectants, UV absorbants, emulsifiers, dispersants, stabilizing agent, coupling agent, cosolvents, compatibility agent, buffering agent, antifoam agent and anti-freezing agent.

Another embodiment of the present invention provides a carrabiitol® formulation for maintaining osmotic balance in plants against abiotic stress, wherein the carrageenan oligosaccharide derivative polyol is obtained from the red seaweed selected from Kappaphycus, Chondrus, Mastocarpus, Halymenia, Porphyra, Solieria, and combinations thereof.

Another embodiment of the present invention provides a carrabiitol® formulation for maintaining osmotic balance in plants against abiotic stress, wherein the red seaweed is more specifically Kappaphycus. The seaweeds are from the coast of Okha, Gujarat.

Another embodiment of the present invention provides a carrabiitol® formulation for maintaining osmotic balance in plants against abiotic stress, wherein the formulation is used for microencapsulation of microbes, and for encapsulation of fertilizers, minerals, agricultural supplements.

Another embodiment of the present invention provides a carrabiitol® formulation for maintaining osmotic balance in plants against abiotic stress, wherein the formulation is effective for any type of plant species when applied to plant, planting material and plant growth supporting substrate”

Another embodiment of the present invention provides a carrabiitol® formulation for maintaining osmotic balance in plants against abiotic stress, wherein the said formulation is water soluble, biodegradable, and supports growth of microorganisms.

Another embodiment of the present invention provides a carrabiitol® formulation for maintaining osmotic balance in plants against abiotic stress, wherein said formulation is converted into the suitable forms selected from sprays, emulsions, suspensions, dusts, powders, pastes and granules, by employing any known mechanical and industrial methods.

Another embodiment of the present invention provides a carrabiitol® formulation for maintaining osmotic balance in plants against abiotic stress, wherein the said abiotic stress is hydric stress, drought, osmotic stress, thermal stress, light stress, nutrient deficiency, and chemical stress generated by metallic or organic pollutant in the soil to grow said plant.

Another embodiment of the present invention provides a carrabiitol® formulation for maintaining osmotic balance in plants against abiotic stress, wherein the formulation promotes plant resistance to abiotic stress is more specifically drought, salinity, extreme temperatures and water stress among others.

Another embodiment of the present invention provides a carrabiitol® formulation for maintaining osmotic balance in plants against abiotic stress, wherein said formulation is used in sustaining plant growth in abiotic stress condition, said plant growth being measured in terms of at least one of: an increased yield, increased seed germination, increased resistant to disease, early maturation, increased leaf area, stem height, leaf width, nitrogen assimilation, protein stabilization, cell division and combinations thereof.

Another embodiment of the present invention provides a carrabiitol® formulation for maintaining osmotic balance in plants against abiotic stress, wherein the said formulation is stable in the wide range of pH 2 to 12, making it amiable for mixing with different agri-inputs for production of combination products and/or for field application in combination with other inputs.

Another embodiment of the present invention provides a carrabiitol® formulation for maintaining osmotic balance in plants against abiotic stress, wherein the said formulation is effective at minimal dosage of 150 ml/acre.

Another embodiment of the present invention provides a method for obtaining carrabiitol® formulation comprising at least one carrageenan oligosaccharide derivative polyol from seaweed(s), wherein the method comprises:
a) processing the seaweed(s) to obtain first residue and first filtrate;
b) diluting the first residue obtained from step (a) with water upto 35% and adjusting the pH in the range of 1 to 5.5 with organic acid and heating at a temperature in the range of 50 to 150° C. for the time period in the range of 30 minutes to 300 minutes to obtain a solution;
c) cooling the solution to ambient temperature and centrifuging/filtering to obtain second residue and second filtrate;
d) treating the second filtrate with a metallic ion complex at a temperature in the range of 30° C. to 110° C. for time period in the range of 30 minute to 300 minutes under continuous stirring followed by neutralising with an alkali solution to obtain a neutralised solution;
e) adding a suitable preservative to the neutralised solution to obtain the carrageenan oligosaccharide derivative polyol; and wherein the carrageenan oligosaccharide derivative polyol_thus obtained has osmo-protectant activity
f) evaporating, drying and sieving the carrageenan oligosaccharide derivative polyol to obtain the carrabiitol® formulation.

It is to be noted that the said osmo-protectant activity is due to the specific conditions and process steps optimised and established. It is this specificity of the optimised conditions and steps of the disclosed process leading to development of carrabiitol formulation that imparts osmo-protectant activity to the said formulation.

Another embodiment of the present invention provides a method for obtaining carrabiitol® formulation comprising at least one carrageenan oligosaccharide derivative polyol from seaweed, wherein the carrabiitol® formulation obtained is almost free of bio-actives and heavy metals; wherein heavy metals are not more than 10 mg/kg.

Another embodiment of the present invention provides a method for obtaining carrabiitol® formulation comprising at least one carrageenan oligosaccharide derivative polyol from seaweed, wherein the carrabiitol® formulation have pH in the range of 4.5 to 7.9.

Another embodiment of the present invention provides a method for obtaining carrabiitol® formulation comprising at least one carrageenan oligosaccharide derivative polyol from seaweed, wherein the carrageenan oligosaccharide is mixed with a specific amount of minerals, beneficial/suitable nutrients for plant growth and/or optionally binding agent to obtain carrabiitol® granule.

Another embodiment of the present invention provides a method for obtaining carrabiitol® formulation comprising at least one carrageenan oligosaccharide derivative polyol from seaweed, wherein the organic acid used in the said method is selected from a group consisting of citric acid, malic acid, ascorbic acid, lactic acid, tartaric acid, oxalic acid, maleic acid, succinic acid, acetic acid, trifluoracetic acid, phosphoric acid, phthalic acid & propionic acid and combinations thereof.

Another embodiment of the present invention provides a method for obtaining carrabiitol® formulation comprising at least one carrageenan oligosaccharide derivative polyol from seaweed, the said organic acid is citric acid, malic acid or lactic acid.

Another embodiment of the present invention provides a method for obtaining carrabiitol® formulation comprising at least one carrageenan oligosaccharide derivative polyol from seaweed, wherein the metallic ion complex is selected from a group consisting of sodium borohydride, potassium borohydride (KBH4), potassium iodide (KI), 4-methyl morpholine borane (MMB), tin chloride, dithionates, thiosulfate, diborane and combinations thereof.

Another embodiment of the present invention provides a method for obtaining carrabiitol® formulation comprising at least one carrageenan oligosaccharide derivative polyol from seaweed, wherein the metallic ion complex is potassium borohydride (KBH4) or potassium iodide (KI) or combinations of both in the range of 0.01% to 10%.

Another embodiment of the present invention provides a method for obtaining carrabiitol® formulation comprising at least one carrageenan oligosaccharide derivative polyol from seaweed, wherein the suitable preservative used in the said method is selected from a group consisting of sodium paraben, methyl paraben, formaldehyde, methanol, potassium sorbate, sodium benzoate and combinations thereof; more preferably formaldehyde or methanol in the rage of 0.01% to 1%.

Another embodiment of the present invention provides a method for obtaining carrabiitol® formulation comprising at least one carrageenan oligosaccharide derivative polyol from seaweed, wherein the said method is a zero discharge method that provides optimum yield, improves extraction efficiency, less energy consumption and maximum utilization of seaweed biomass as well as economical and viable process.

Another embodiment of the present invention provides a method for obtaining carrabiitol® formulation comprising at least one carrageenan oligosaccharide derivative polyol from seaweed, wherein the obtained formulation has a shelf-life of at least three years.

Another embodiment of the present invention provides a method for obtaining carrabiitol® formulation comprising at least one carrageenan oligosaccharide derivative polyol from seaweed, wherein the obtained formulation is clear and transparent, non-crystalline and stable at temperatures in the range of 1° C. to 60° C.

Another specific embodiment of the present invention provides a method for extraction of oligosaccharide polyols from seaweed by biomass solubilisation, hydrolysis and its further reduction with reducing agent.

In yet another embodiment the carrabiitol® formulation comprises the seaweed based oligosaccharide polyols. The said formulation comprises carrageenan oligosaccharide polyols and its derivatives that functions similar to osmolytes with antioxidant.

According to another embodiment of the specification the carrabiitol® formulation obtained is completely free of bio-actives and has heavy metals not more than 10 mg/kg.

According to yet another embodiment of the specification the carrabiitol® granules are obtained by mixing carrabiitol® formulation with a specific amount of minerals, biomass, beneficial /suitable for plant growth.

According to another embodiment of the specification the carrabiitol® formulation pH range is 4.5 to 7.9.

According to an embodiment of the present invention the Carrabiitol® formulation can further comprise other active compounds against pests, such as insecticides, herbicides, fungicides; herbicidal or growth-regulating active compounds.

Another embodiment of the present invention recites the use of the said carrabiitol® formulation for soil amendment purposes, wherein the carrabiitol® formulation comprise one or more fillers/carriers and/or optionally binding agent to make granules to make granules. Since carrabiitol® formulation has viscous properties, it coagulates with the carrier agent and forms granules without any added manufacturing cost. These carrabiitol® granules, thus formed, can be employed for different purposes. Few of examples are follows:
These carrabiitol® granules can be used as soil conditioners, as they will increase soil water holding capacity and also provide organic substrate which helps in absorption of plant nutrients.
Carrabiitol® granules can also be used as carrier for even trace elements or other beneficial component.
Carrabiitol® granules also increase plant basal metabolism, nitrogen assimilation and cell division.

In yet another specific embodiment of the specification the said formulation is used for microencapsulation of microbes, and encapsulation of fertilizers, minerals, agricultural supplements and other biostimulants.

Yet another specification provides for the application of the formulation by priming and/or coatings of seeds.

In another embodiment the said formulation is effective for any type of plant species.

An embodiment of the invention relates to the plants to be treated by the said invention. These comprises agricultural, silvicultural, ornamental and horticultural plants. The plants to be treated by the said invention may include plants that can be harvested or cultivated on a commercial scale or which serve as an important source of feed, food, fibres, fuel or sources of fuels (e.g. wood, bioethanol, biodiesel, biomass) or sources of other chemical compounds The plants to treated by the said invention can be natural or wild types or modified by conventional or molecular breeding methods, mutagenesis or genetically modified.

In a particular embodiment the abiotic stress tolerance in plants is salt tolerance, drought tolerance and water tolerance.

One specific embodiment of the present invention relates to inducing low temperature abiotic stress tolerance in the plants by use of carrabiitol® formulation

Further, the said formulation work as ROS detoxifiers (antioxidants), photosynthesis protectants, osmo-protectants, macro-biomolecule stabilizers, and protein folding enhancers.

According to an embodiment of the specification the said carrabiitol® formulation is applicable at different stages of plant life cycle. The formulation can be used as a seed coating or seed priming to overcome stress. Similarly the carrageenan oligosaccharide derivative polyols based formulation enables enhancement of plant metabolism. Therefore, it can also be used as foliar spray at plant vegetative stage as well as flowering stage. The formulation enhances efficient carbon use and fruit maturation and can further be used during fruiting periods.

An embodiment of the invention refers to the use of the formulation as an effective soil amendment agent. The formulation, carrabiitol® contains oligosaccharide derivative polyols and therefore is also a nutrient (energy) source for soil microflora. It also act as antioxidant defence molecule and metal chelator. Therefore, the formulation can be composed in a manner to be used as an effective soil amendment.

An embodiment of the invention refers to the use of the formulation as an effective soil amendment agent.

According to yet another embodiment the said extraction procedure gives optimum yield, improves extraction efficiency, has less energy consumption and maximum utilization of seaweed biomass and is an economical as well as viable process. Further the said extraction procedure is Zero Discharge Process and does not involve any flammable, hazardous and carcinogen chemicals or solvents making it safe and environment friendly process.

According to yet another embodiment of the specification the said formulation is water soluble and biodegradable and is utilized by plants for generating ATP via Glycolysis pathway. Further the said formulation supports the growth of microorganism thus making it suitable for applications with microbial products.

As stated above, the carrabiitol® formulation comprising the seaweed based polyols are used in “effective amounts”. This means that they are used in a quantity which allows to obtain the desired effect which is a synergistic increase of the health of a plant but which do not give rise to any phytotoxic symptom on the treated plant.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may vary.

### 1. Extraction and Preparation of Carrabiitol Using Malic Acid and Potassium Iodide

Collected fresh seaweed and immediately was processed to remove salts, sands and other impurities. After that obtained seaweed immediately was subjected to remove bio-actives contain seaweed sap by cell stretching system and collected residue for furthered process. The seaweed sap thus obtained is utilized separately for manufacturing of bio-stimulant and fertilizer preparations. Fresh seaweed was processed due to crystallization of salts or reabsorption of salts occurs in dried seaweed biomass or longer storage biomass, which leads to process harder to collect bio-actives free residue.

40 g dried biomass of K-carrageenan was dissolved in 1000 ml of water and analyzed for pH. Added malic acid to bring its pH 1 to 3. The solution was stirred to dissolve and heated the mixture at 80-150° C. for 3 to 5 hrs. After heating, the solution was cooled and centrifuged. The filtrate was taken and added upto 5% potassium iodide and heated the solution with continuous stirring at 50 to 110° C. for 2 to 5 hrs. Sodium hydroxide or potassium hydroxide was added for neutralization. The suitable preservative was added and filled the formulation into bottles and stored at Room temperature for future analysis. The insoluble residue collected was subjected to process of making cellulose film by known method.

The above formulation was evaporated to reduce volume to its half and oven dried for required powder form. Obtained powder was passed from 100 mesh sieve to make uniform mixture and stored in sample bag at room temperature for future analysis.

### 2. Characterization of Carrabiitol

### A. HPLC Analysis

RP- HPLC profiling was carried out with RI detector using Phenomenex Rezex RPM monosaccharide PB+ column at 80° C. and deionized water as mobile phase.

Before injecting, samples were filtered through 0.45 µ filter membrane.

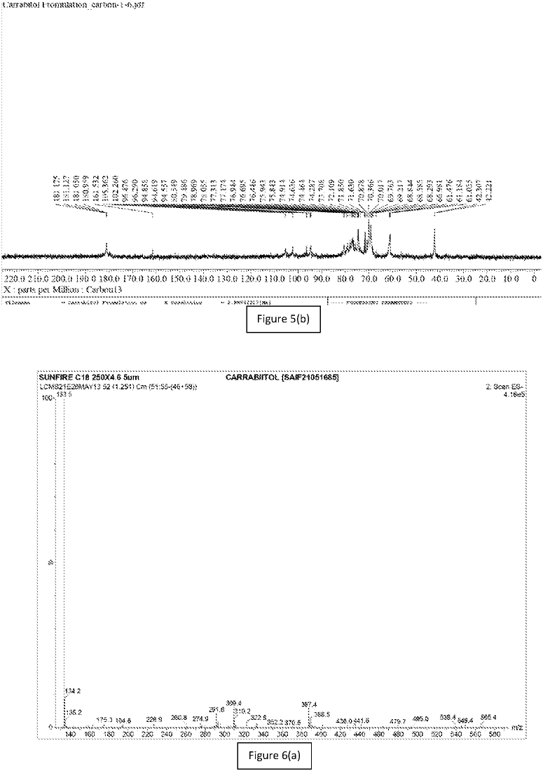

HPLC profiling showing that carrabiitol formulation has comprising four derivatives of carrageenan oligosaccharides polyols (FIG. 4).

### B. Mass Spectrometry Analysis

For nuclear magnetic resonance spectroscopy,¹H NMR and ¹³C NMR Spectrum were obtained with a Bruker advance 600 MHz spectrometer operating at 600.17 MHz and 150.91 MHz for ¹H and ¹³C respectively. Samples were prepared in D20 (99%) and spectrum recorded. Chemical shift reported in parts per millions (PPM).

¹H and ¹³C NMR Spectrum of carrabiitol confirms the presence of derivatives of carrageenan oligosaccharide polyol. In ¹³C NMR, the chemical shifts from 105.362 ppm to 42.221 ppm is due to G4S (D-galactose 4 Sulfate) and DA (3, 6 anhydro D-galactose) structure of derivatives of oligosaccharide alditol. The chemical shift in ¹H NMR at 2 to 4 ppm chemical shift is due to alcohol group present at the reducing end of G4S terminal.

LC-MS and ESI-MS were carried out for acute molecular weight identification of obtained compound. Detection were performed in negative ion mode as well as positive ion mode coupled with Agilent 6520 (Q-TOF) Mass spectrometer with Agilent 1200 HPLC system.The molecular weight was calculated based on mass to charge ratio called “Mathematical Charge Deconvolution”. The composition of carrageenan oligosaccharide derivatives polyols obtained was presented in table 1.

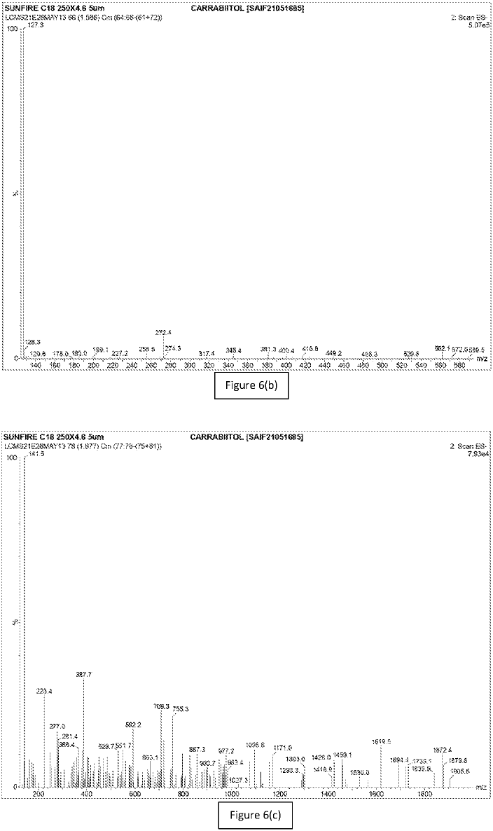

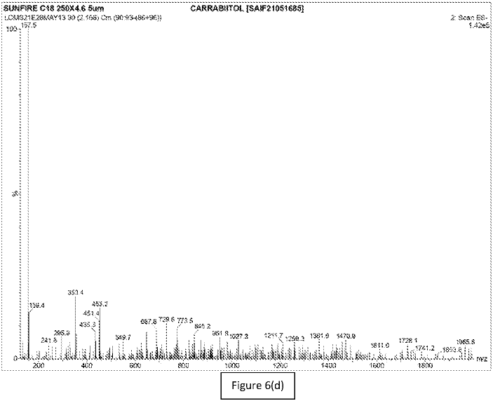

The NMR Spectrum, LC-MS (ESI-MS) mass spectrum were presented in FIG. 5 & FIG. 6 respectively.

TABLE 1

Assignment of ion composition of LC-MS compounds and ESI-MS Spectra of Carrageenan oligosaccharide polyols

| Sr. no. | Ion Composition | Carrageenan oligosaccharide derivative polyols | Calculated Mol. weight (Dalton) |
|---|---|---|---|
| 1 | [M-Na]+ | Dimer | 556.593 |
| 2 | [M-Na]+ | Trimer | 1940.47 |
| 3 | [M-Na]+ | Tetramer | 4501.596 |
| 4 | [M-Na]+ | Hexamer | 10718.155 |

### 3. Extraction and Preparation of Carrabiitol Using Citric Acid and Sodium Borohydride

20 g dried biomass of K-carrageenan was dissolved in 500 ml of water and analyzed for pH. Added citric acid to bring its pH 1 to 3. Solution was dissolved and heated the mixture at 80-150° C. for 3 to 5 hrs. After heating, the solution was cooled and centrifuged. Filtrate was taken and added 2% Sodium borohydride and heated the solution with continuous stirring at 50-110° C. for 2 to 4 hrs. Sodium hydroxide or potassium hydroxide was added for neutralization. Suitable preservative was added and filled the formulation into bottles and stored at room temperature for future analysis. The insoluble residue collected was subjected to process of making cellulose film by known method.

The above formulation was evaporated to reduce volume to its half and oven dried to get powder form. Obtained powder was passed through 100 mesh sieve to make uniform mixture and stored in sample bag at room temperature for future analysis.

### 4. Method for Preparation of Carrabiitol® granules

200 ml of prepared Carrabiitol formulation was mixed with 320 g of lignite and oven dried at a temperature of 64° C. for 6-8 hrs. Irregular shaped carrabiitol granules were obtained and analysed for its water and liquid holding capacity by standard methods. Results were shown in table 2.

TABLE 2

Water holding capacity of carrabiitol

| Sr No | Parameter Name | Result (% w/w) |
|---|---|---|
| 1 | Water Holding Capacity | 25-30% |
| 2 | Liquid Holding Capacity | 15-20% |
| 3 | Potassium | 0.5 to 1% |

As observed from table 2, liquid holding capacity of the granules was 15-20%, which was at par with commercially available samples. It indicates that these carrabiitol granules can also be used as carrier of trace elements or any other active ingredients.

### 5. Stability Study of Carrabiitol Liquid Formulation, Carrabiitol Powder & Carrageenan Oligosaccharide Mixture

The samples of carrabiitol liquid formulation (Batch no. 41/L20 MFG dated December 2020), carrabiitol powder and carrageenan oligosaccharides were taken for stability study as per CIPAC MT 46.1. Sample taken after shaking well and analyzed for pH physical observation and HPLC Run by validated Analytical Method. Bottles were kept at 54° C. ± for 42 days. After storage periods, the bottles were cooled for 24 hr at room temperature and analyzed for physical observation and HPLC run.

After stability study, physical observations of carrabiitol formulation and carrabiitol powder are acceptable as per requirement of STD Certified Limits ±% (40CFR 158.350(b) (2), whereas the carrageenan oligosaccharide separated out with two different layers.

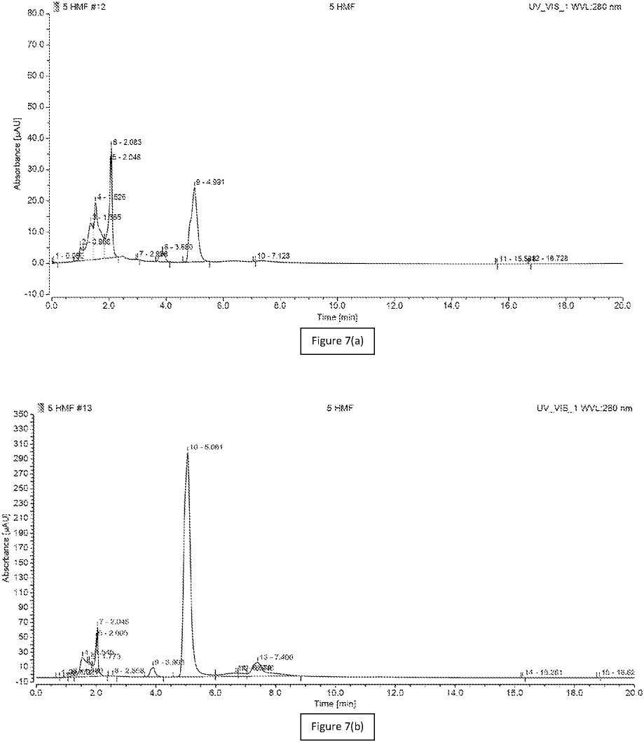

The HPLC run of both formulation were carried out and presented in FIG. 7.

The physical parameters of initial sample and sample analysed after 42 days of storage at accelerated conditions of temperature were found similar, indicating that gross physical characteristics of carrabiitol powder does not produce any significant changes. The above study indicates that carrabiitol powder was stable at ambient temperature for more than three year. Same experiment was carried out with carrabiitol liquid formulation. Result indicate that carrabiitol formulation was stable at ambient temperature for more than 3 years.

The HPLC chromatogram of carrageenan oligosaccharide before and after stability (FIG. 7) showed that the one compound was converted into another compound after 42 days of accelerated storage at 54° C. It indicates that said formulation was not stable, whereas in carrabiitol formulation HPLC chromatogram was similar before and after accelerated storage period, which was clearly identify that carrabiitol was stable. Stability parameters of carrabiitol formulation is provided in table 3.

TABLE 3

Stability parameters of carrabiitol formulation at 54° C. for 42 days
Result of carrabiitol formulation after stability studies at 54° C. for 42 Days

| Sr no. | Parameter | Before Stability (9-3-2021) | After Stability (21-4-2021) | STD Certified Limits ± % (40 CFR 158.350(b)(2).) | Difference | Accepted (Y/N) |
|---|---|---|---|---|---|---|
| 1 | Physical appearance | Golden yellow colour transparent liquid | Golden yellow colour transparent liquid | NA | - | - |
| 2 | Phase separation | No phase separation | No phase separation | NA | - | - |
| 3 | Clumping | No | No | NA | - | - |
| 4 | Weight of sample in g | 102.69 | 102.01 | 2 | 0.68 | Y |
| 5 | PH | 6.577 | 6.427 | 5 | 0.15 | Y |
| 6 | Density g/ml | 1.0269 | 1.0113 | 2 | 0.0156 | Y |
| 7 | 3 g/L Solution appearance | Clear transparent liquid | Clear transparent liquid | NA | - | - |

### 6. Nutrient Source for Soil Microflora

Prepared carrabiitol Formulation was mixed with nutrient agar and sterilized in Autoclave at 121° C. for 20 minutes with 15 Ib pressure. Plates were prepared as per standard method for incubation. Soil was collected from farm and proceeded for colony forming unit per gram or CFU estimation, with and without carrabiitol formulation. Results are shown in table 4 below.

TABLE 4

Nutrient availability from Carrabiitol granules

| Sr No. | Treatment | Cfu/g |
|---|---|---|
| 1 | Nutrient agar without Carrabiitol Formulation | 4.5 × 106 |
| 2 | Nutrient agar with Carrabiitol formulation | 6.7 × 107 |

As was evident from Table 4 that soil microflora can utilize carabiitol formulation as their nutrient source which ultimately helps in soil conditioning and leads better growth of plants. Soil cfu/g is more in plates with carrabiitol formulation than in nutrient agar only. This is likely due to higher concentration of nutrient available from carrabiitol formulation. (Data Courtesy: Microbiology Department, Agri Biochem Research Lab, Panoli)

### 7. Abiotic Stress Studies

### I. Abiotic Bioassay for Drought Stress

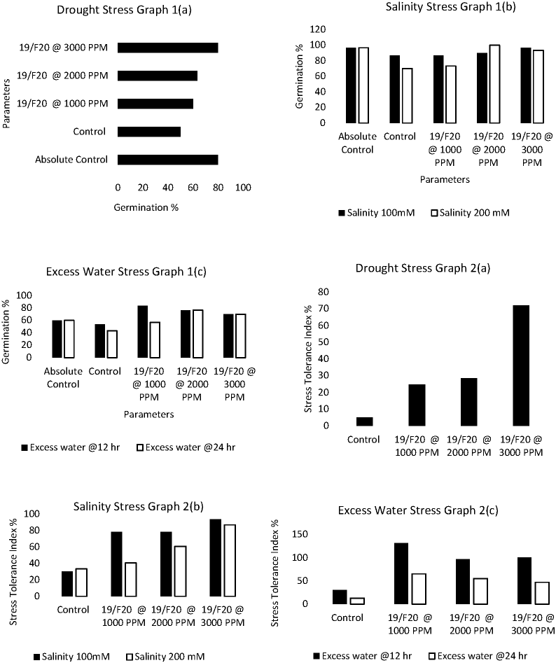

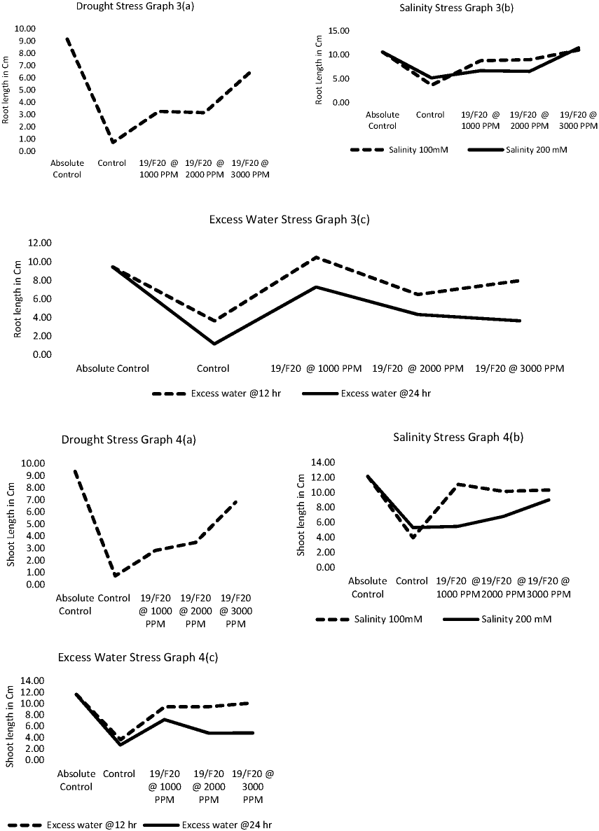

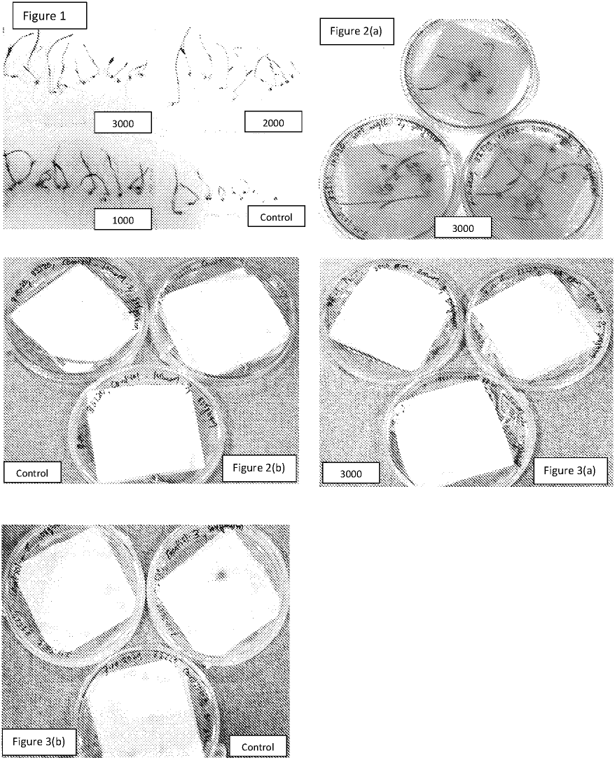

Wheat, sorghum and fenugreek seeds were collected from agriculture Seed shop in Gujarat. Seeds were surface sterilized and transferred them into petri-plate containing 600 mg/l & 1000 mg/l carrabiitol formulation and allowed to equilibrate for 12 hours, along with control. Water was used as control. Shed dried them and transferred into germination plates. Drought stress was created by PEG solution (25%) at three days interval up to 15 days. (See FIGS. 1 and 2) All experiments were carried out under controlled conditions.

Results were shown in table 5, table 6 and table 7.

TABLE 5

Wheat seeds assay

| Parameter | Absolute Control (water and w/o stress) | Negative Control (PEG stressed & w/o Carrabiitol formulation) | 600 mg/L (Carrabiitol Formulation) | 1000 mg/L (Carrabiitol Formulation) |
|---|---|---|---|---|
| Germination % | 64.29 | 35.71 | 78.57 | 71.43 |
| Root length in cm | 6.12 | 0.5 | 7.33 | 9.6 |
| Shoot length in cm | 12.5 | 1.2 | 3.62 | 12.33 |
| Vigor index % | 1197.32 | 60.71 | 860.75 | 1566.43 |
| Stress tolerance index % | NA | 5.07 | 71.89 | 130.83 |

TABLE 6

Sorghum seeds assay

| Parameter | Absolute Control (water and w/o stress) | Negative Control (PEG stressed & w/o Carrabiitol ) | 600 mg/L | 1000 mg/L |
|---|---|---|---|---|
| Germination % | 100 | 71.43 | 85.71 | 85.71 |
| Root length in cm | 5.75 | 1.0 | 5.83 | 11.13 |
| Shoot length in cm | 3.75 | 1.0 | 1.0 | 5.25 |
| Vigor index % | 950 | 142.86 | 585.46 | 1403.57 |
| Stress tolerance index % | NA | 15.04 | 61.62 | 147.74 |

TABLE 7

Fenugreek seeds assay

| Parameter | Absolute Control (water and w/o stress) | Negative Control (PEG stressed & w/o Carrabiitol) | 600 mg/L | 1000 mg/L |
|---|---|---|---|---|
| Germination % | 100 | 71.43 | 85.71 | 92.86 |
| Root length in cm | 1.0 | 0.50 | 1.00 | 2.50 |
| Shoot length in cm | 0.5 | 0.20 | 0.58 | 0.50 |
| Vigor index % | 150 | 50 | 135.43 | 278.57 |
| Stress tolerance index % | NA | 33.33 | 90.29 | 185.71 |

As per the above results it is noticed that use of carrabiitol formulation enhances the germination percentage and also increases the growth of shoot and root under stressed drought conditions in wheat, sorghum and fenugreek seeds. It also observed that the tolerance of stress condition in carrabiitol formulation coated seeds was increased compared to negative control.

### 8. Seed Priming of Sorghum Seeds With Carrabiitol® Increases Germination Rate and Stress Tolerance Index Under Abiotic Stress Conditions i.e. Salinity Stress, Drought Stress and Excess Water Stress

### Materials & Methods

The experiment was carried out as laboratory study at Agri BioChem Research lab, Panoli, Gujarat. Seeds of KAVERI COLONEL 6363 sorghum was collected from Agriculture Shop in Gujarat. Three different abiotic stress studies were carried out including drought, excess water and salinity stress with different concentration of carrabiitol. The standard error of mean+_ and Critical difference is calculated at 95% confidence level with p value <0.05.

Seeds were surface sterilized and soaked for 16 hr in carrabittol formulation, (Batch no.19/F20) @1000 ppm, @2000 ppm @3000 ppm, @ Absolute control, Water and only respective stress caused solution @ Control. After that, seeds were shed drying for 4 hrs and transferred in petri-plates. All experiments were carried out in triplicates. Seeds were put at 25° C. for 15 days by maintaining 16/8 h day/night light with intensity of 400 micromole/ms. Seeds were grown by injecting 5 ml solution of water or stress solution at three days interval. After germination experiments, root length, shoot length, germination percentage were calculated The vigour index was calculated using the formula proposed by Abdul-Baki and Anderson (1973) and the stress tolerance index was calculated using the formula proposed by Dhopte and Livera (1989) and expressed as percent.

### Result

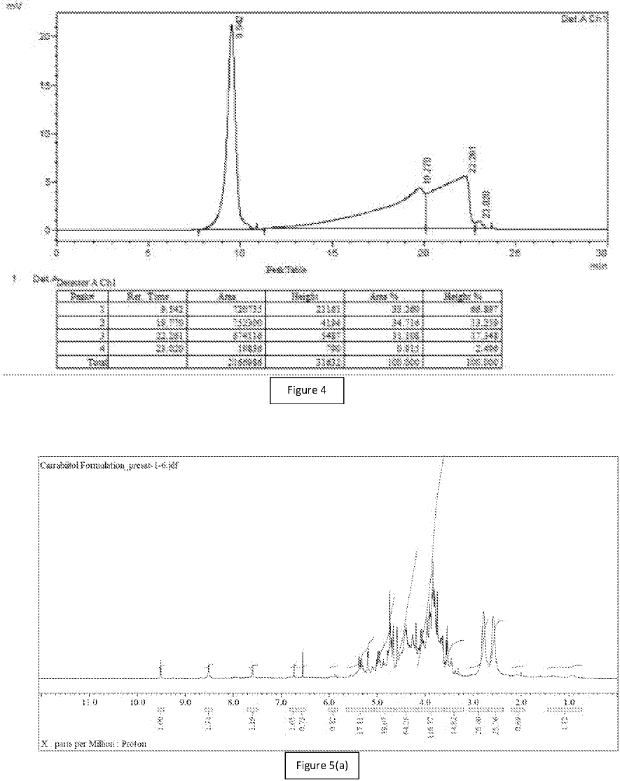

Results showed that sorghum seeds treated with carrabiitol® have about 15-20% higher germination rate compare to control in all abiotic stress condition i.e. water stress, salinity stress and drought stress. Data presented in table 8, 9, and 10 & FIGS. 1, 2 and 3

TABLE 8

SEM & CD value of all parameters of sorghum under drought stress condition
Drought Stress

| Treatment Name | Germination % | RL (cm) | SL (cm) | STI (%) |
|---|---|---|---|---|
| Absolute Control (T-1) | 80.00 | 9.17 | 9.37 | NA |
| Control (T-2) | 50.00a* | 0.73a* | 0.73a* | 5.76a* |
| CB 1000 mg/L (T-3) | 60.00 | 3.27 | 2.83 | 25.51 |
| CB 2000 mg/L (T-4) | 63.33 | 3.17 | 3.50 | 29.01 |
| CB 3000 mg/L (T-5) | 80.00a* | 6.50a* | 6.83a* | 71.42a* |
| Sem(+/-) | 6.36 | 0.84 | 0.8 | 8.07 |
| CD(0.05) | 20.76 | 2.74 | 2.6 | 27.93 |

*a= Significantly differ, p<0.05, CB (Carrabiitol) RL (Root length), SL (Shoot length), STI (Stress tolerance index)

TABLE 9

SEM & CD value of all parameters of sorghum under Salinity stress condition
Salinity Stress

| Treatment Name | Germination % | RL (cm) | SL (cm) | STI (%) |
|---|---|---|---|---|
| Absolute Control (T-1) | 96.67 | 10.60 | 12.17 | NA |
| Control (T-2)100 mM | 86.67 | 3.70a* | 4.00a* | 30.46a* |
| CB 1000 mg/L (T-3) | 86.67 | 8.80 | 11.10a* | 78.13 |
| CB 2000 mg/L (T-4) | 90.00 | 9.00a* | 10.17 | 78.09 |
| CB 3000 mg/L (T-5) | 96.67 | 5.17 | 5.33 | 93.73a* |
| Control (T-6)200 mM | 70.00a* | 6.67a* | 5.50 | 32.82a* |
| CB 1000 mg/L (T-7) | 73.33 | 6.53 | 6.83 | 40.21 |
| CB 2000 mg/L (T-8) | 100.00a* | 11.47a* | 9.03a* | 60.50 |
| CB 3000 mg/L (T-9) | 93.33a* | 11.00a* | 10.37a* | 88.24a* |
| Sem(+/-) | 3.51 | 0.97 | 0.82 | 7.53 |
| CD(0.05) | 10.53 | 2.92 | 2.46 | 22.84 |

*a= Significantly differ, p<0.05, CB (Carrabiitol) RL (Root length) SL (Shootlength), STI (Stress tolerance index), CD (Critical Difference)

TABLE 10

SEM & CD value of all parameters of sorghum under excess water stress condition
Excess Water Stress

| Treatment Name | Germination % | RL (cm) | SL (cm) | STI (%) |
|---|---|---|---|---|
| Absolute Control (T-1) | 60.00 | 9.47 | 11.67 | NA |
| Control (T-2) | 43.33a* | 1.17a* | 2.70 | 30.23a* |
| CB 1000 mg/L (T-3) | 56.67 | 7.30a* | 7.20 | 130.20a* |
| CB 2000 mg/L (T-4) | 76.67a* | 4.33 | 4.80 | 94.24 |
| CB 3000 mg/L (T-5) | 70.00a* | 3.67 | 3.67 | 100.28a* |
| Control (T-6)200 mM | 53.33a* | 3.67a* | 3.67a* | 15.32a* |
| CB 1000 mg/L (T-7) | 83.33a* | 10.50a* | 9.47 | 66.11 |
| CB 2000 mg/L (T-8) | 80.00a* | 6.50 | 9.50a* | 55.41 |
| CB 3000 mg/L (T-9) | 70.00 | 8.00 | 10.17a* | 47.60 |
| Sem(+/-) | 6.18 | 1.59 | 1.93 | 19.76 |
| CD(0.05) | 18.54 | 4.79 | 5.81 | 59.96 |

*a= Significantly differ, p<0.05, CB (Carrabiitol) RL (Root length) SL(Shoot length), STI (Stress tolerance index), CD (Critical Difference)

From above experiments and results it was concluded that carrabiitol formulation gives 30% to 50% effective results to combat abiotic stress, even it at extreme condition. Higher stress tolerance index also indicates that carrabiitol formulation work as Osmolyte. FIG. 1 shows comparison of root and shoot length in excess water stress condition in sorghum.

### 9. Dicot Seeds Treatment With Carrageenan Oligosaccharide Derivative Polyols Increase Germination Rate and Stress Tolerance Index Under Salinity Stress

### Materials and Methods

Fenugreek (Trigonella foenum-graecum) seeds of variety MAHER-1 incubated with carrageenan oligosaccharide derivative polyols@ 1000 ppm, @2000 ppm @3000 ppm, @ Control for 16 hr of period along with water (Absolute Control). Seeds were than collected and shed drying for 4 hrs. The salinity stress was induced by 100 mM and 200 mM of NaCl solution. Experiments were carried out in triplicates. Seeds were put at 25° C. for 15 days by maintaining 16/8 hrs day/night light with intensity of 400 micromole/ms. After germination experiments, root length, shoot length, germination percentage were calculated The vigour index was calculated using the formula proposed by Abdul-Baki and Anderson (1973) and the stress tolerance index was calculated using the formula proposed by Dhopte and Livera (1989) and expressed as percent.

### Result

Results showed that fenugreeks seeds treated with carrageenan oligosaccharide derivative polyol have about 10% higher germination compare to Control. Similarly, it is also showed that stress tolerance index is also about 21-30% higher in seeds treated with carrageenan oligosaccharide derivative polyols.

It indicates that carrageenan oligosaccharide derivative polyol composition is effective on both types of plants i. e. mono cot and dicot under abiotic stress condition.

### 10. Foliar Spray of Carrabiitol Increase Relative Water Content and Proline Content in Wheat Plant Under Field Condition

Three month field experiment (JAN 2021 to March 2021) was conducted at the Village of Kharod, Taluka Ankleshwar, Gujarat, India The soil was deep and well drained, loamy in nature. Triticum aestivum (Wheat) of sharbati tukri variety selected for field trial experiment. The experiment was laid down with 16 ×16 m2 area. Soil was irrigated with Urea and sulfate. The Fertigation practice followed as per traditional requirement in both control and carrabiitol formulation. Metsulfuron methyl 20% WP & Sulfosulfuran 75% + Metsulfuron 5% WG used for weed and insect control management. Foliar application of 0.3% carrabiitol formulation carried out on 50 days old wheat and after that 15 days of interval upto three application. At physiological maturity stage stem height, leaf length, leaf width and number of buds, leaf dry weight, relative water content %, Chlorophyll content (Arnon,et al, 1949) and proline content were observed. At the harvesting stage, collect 1 m2 × 1 m² area of wheat plants and weight it in triplicates.

The observation after physiological development of wheat are shown in table 11.

TABLE 11

Physical parameters observation after third application of carrabiitol
Observation after 3rd application of Carrabiitol Dated 19022021

| Sr No. | Control | | | | Carrabiitol Formulation | | | |
|---|---|---|---|---|---|---|---|---|
| | Plant Height in cm | Leaf length in cm | No of Buds | Leaf Width in cm | Plant Height in cm | Leaf length in cm | No of buds | Leaf Width in cm |
| 1 | 94.5 | 24 | 15 | 1.5 | 95.5 | 25.5 | 16 | 1.9 |
| 2 | 88.12 | 24.6 | 15 | 1.8 | 95.5 | 24.3 | 18 | 1.6 |
| 3 | 87.5 | 23.5 | 17 | 1.5 | 89 | 24.6 | 18 | 1.9 |
| 4 | 82 | 23 | 17 | 1.6 | 91.5 | 23.9 | 19 | 1.8 |
| 5 | 94 | 27 | 18 | 1.9 | 88.9 | 28.5 | 19 | 1.4 |
| 6 | 85.5 | 24.6 | 18 | 1.6 | 91 | 27.9 | 19 | 1.9 |
| 7 | 98 | 23 | 18 | 1.4 | 105 | 25.61 | 18 | 1.8 |
| 8 | 94.5 | 24 | 18 | 1.5 | 92.5 | 26.9 | 17 | 1.6 |
| 9 | 89 | 27.5 | 18 | 1.8 | 102 | 24.9 | 18 | 2 |
| 10 | 82 | 28 | 17 | 1.9 | 90.5 | 26.7 | 17 | 1.6 |
| Average | 89.51 | 24.92 | 17.1 | 1.65 | 94.14 | 25.881 | 17.9 | 1.75 |
| SE (Standard Error) | 1.665 | 0.564 | 0.359 | 0.055 | 1.643 | 0.468 | 0.298 | 0.057 |
| % of effectiveness | | | | | 5.17 | 3.86 | 4.68 | 6.06 |

TABLE 12

Chemical parameters of wheat leaf’s after third application of carrabiitol formulation
Observation of chemical Parameters after 3rd Application of Carrabiitol Dated 19022021

| Control | | | | | | | | | Carrabiitol | | | | | | | |
|---|---|---|---|---|---|---|---|---|---|---|---|---|---|---|---|---|
| Sr no. | Chlorophyll content mg/kg | | Relative water Content % w/w | | Proline content µM/g fresh weight | | Leaf Dry matter % w/w | | Chlorophyll content mg/kg | | Relative water Content % w/w | | Proline content µM/g Fresh weight | | | Leaf Dry matter % w/w |
| 1 | | 58.56 | | 44.12 | | 0.765 | | 32.62 | | 136.3 | | 54.23 | | 0.903 | 32.94 | |
| 2 | | 166.52 | | 56.72 | | 0.771 | | 32.86 | | 104.01 | | 60.45 | | 0.859 | 33.59 | |
| 3 | | 118.93 | | 47.91 | | 0.773 | | 31 | | 105.1 | | 57.92 | | 0.888 | 37.92 | |
| Average | | 114.67 | | 49.58 | | 0.77 | | 32.16 | | 115.14 | | 57.53 | | 0.88 | 34.82 | |
| SE (Standard Error) | | 25.506 | | 3.047 | | 0.002 | | 0.477 | | 8.644 | | 1.475 | | 0.011 | 1.276 | |
| % of effectiveness | | | | | | | | | | 0.41 | | 16.03 | | 14.76 | 8.26 | |

The physical parameters observation represent that carrbiitol formulation was effective at 3 to 6%. The chemical analysis Data showed that carrabiitol formulation was effective in the range of 10% to 16% for relative water content %, proline content and leaf dry matter %. The higher percentage of relative water content in carrabiitol treated plants indicated that plant developed ability to combat abiotic stress condition. Same higher concentration of proline content compare to control represent that said formulation work as osmolyte and activate stress signaling pathway, which leads to release higher percentage of proline content. As accumulation of proline in plants was known to have both osmo-protectory and antioxidant functions, results show that carrabiitol enhance osmo-protection and protection against oxidative damage of the plant.

The Experiment results also showed that carrabiitol which contains only carrageenan oligosaccharide derivative polyols are compatible with other Fertilizers and pesticides also i.e. conventional agriculture treatment scheme.

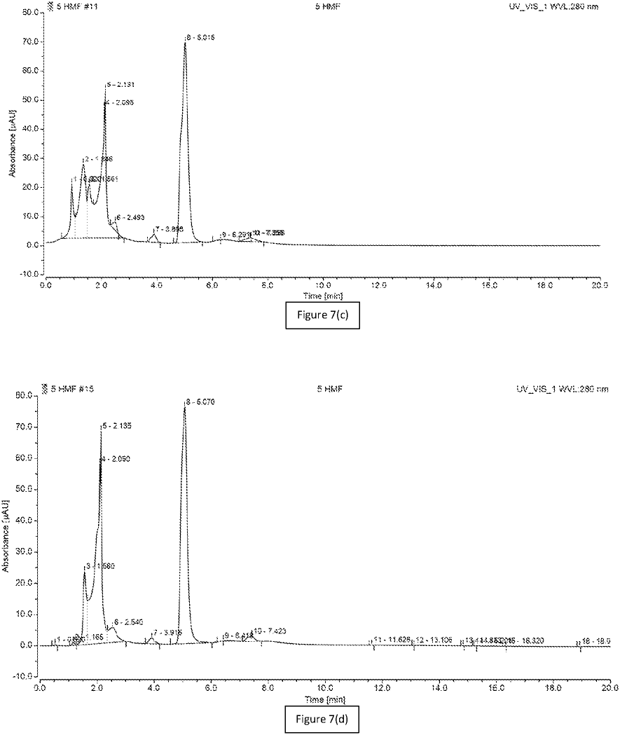

At the harvesting point, the parameters included grain yield%, 100 grain weight, one kernel contain buds observed. The data of observation indicated that carrabiitol formulation is effective at 3000 mg/l and effectiveness range is 10% to 25%. Wheat kernel and biomass of control and carrabiitol represent in FIG. 8.

TABLE 13

Observation of carrabiitol treated Wheat plants at harvesting phase
Observations at Harvesting

| Sr No. | Weight of dried plant on 1 m2 area | | Weight of 75 dried plants in g | | Weight of 10 buds containing fruits in g | | Weight of 100 Wheat in g | |
|---|---|---|---|---|---|---|---|---|
| | Control in kg | Carrabiitol in Kg | Control in g | Carrabiitol in g | Control in g | Carrabiitol in g | Control in g | Carrabiitol in g |
| 1 | 3.011 | 2.24 | 158 | 243 | 21.88 | 24.68 | 4.6192 | 4.0029 |
| 2 | 2.24 | 2.611 | 195 | 209 | 21.9 | 24.1 | 4.5215 | 4.8886 |
| 3 | 2.46 | 3.405 | 219 | 213 | | | | |
| Average | 2.570 | 2.752 | 190.667 | 221.667 | 21.890 | 24.390 | 4.570 | 4.896 |
| SE (Standard Error) | 0.187 | 0.281 | 14.486 | 8.760 | 0.007 | 0.205 | 0.035 | 0.005 |
| % of Effectiveness | | 7.068 | | 16.259 | | 11.421 | | 7.120 |

The above result shows that carrabiitol treated plants gives healthier wheat and greater biomass compare to control. It indicates the greater yield percentage. The kernel of one plant contain wheat is also recorded in ten replicates and results are below in table 14. Result shows that buds on kernel is more than control, which ultimately gives higher production of wheat per acre at the farm. The overall effectiveness of carrabiitol formulation on bud production is 13.65%

TABLE 14

Number of buds on one kernel of wheat

| Sr No. | Wheat on one bud | |
|---|---|---|
| | Control | Carrabiitol |
| 1 | 26 | 36 |
| 2 | 39 | 35 |
| 3 | 32 | 49 |
| 4 | 28 | 30 |
| 5 | 42 | 36 |
| 6 | 32 | 49 |
| 7 | 40 | 43 |
| 8 | 26 | 26 |
| 9 | 37 | 36 |
| 10 | 35 | 43 |
| Average | 33.700 | 38.3 |
| SE (Standard Error) | 3.194 | 4.164 |
| % of Effectiveness | | 13.650 |

### 11. Foliar Spray of Carrabiitol on Tender Crops to Evaluate Physical Health of Plants

Experiment was carried out to evaluate physical effects of tender crop at the application rate of 150 L/ Acre on various Location of Madhya Pradesh (MP), India at the 3000 mg/L concentration. The tender crop selected for experiment were Garlic, Cucumber, Okra, Gram, Capsicum, Marry gold and tomato. Physiological observations carried out after 15 days of application. Farmer reviews were also recorded.

The general physical effect on plant health under treatment of carrabiitol formulation on tender crops are listed below in table 15.

TABLE 15

Physical effects on plant health under treatment of carrabiitol on tender crops and farmer review

| Sr no. | Crop name | Date of application | Date of observation | Observation | Farmer’s review |
|---|---|---|---|---|---|
| 1 | Garlic | 20-01-2021 | Sep. 02, 2021 | Healthier plant, increase greeneries and height | Healthier plant and increase height |
| 2 | Cucumber | 16-02-2021 | 22-02-2021 | Compare to other plants result is much better of carrabiitol treated plant | Increase in leaf width, Plant height, more branches, higher greeneries |
| 3 | Okra | 17-02-2021 | 23-02-2021 | Healthier plant, increase greeneries and height | Healthier plant and increase height |
| 4 | Gram | 18-02-2021 | 25-02-2021 | Healthier plant and more flowers | Increase height, Greeneries, more flowers and branches |
| 5 | Capsicum | 18-02-2021 | 25-02-2021 | More branches, more flower and healthier plants | More greeneries, branches and flowers. |

The observation and farmer reviews shows that from above table, the carrabiitol formulation gives significantly greater results and its effectiveness at desired concentration.

The results also represent that carrageenan oligosaccharide derivative polyols improves plant health, even under non stressed conditions by maintaining osmotic balance.

### 12. Cold Test and Dilution Stability Study of Carrabiitol Formulation

The sample of carrabiitol formulation (Batch no. 41/L20) was taken for cold test analysis as per IS 6940:1982 RA 2017. Take 50 ml of the material into 100 ml container and close it with a stopper fitted with thermometer. Cool the material to 10° C. by placing in ice-cold water. Gently stir the material in the container at short intervals for 1 hour maintaining the temperature of the material at 10° C. At the end of one hour, examine the sample for turbidity, or separated solid. Same test was carried out at 5° C.

For dilution stability study, CIPAC MT 41 reference method was carried out. Dilute the sample with HPLC water as such that sample concentration reached 0.3%, and allow the solution to stand at 20° C. for 18 hrs. At the end of this time, observe for any separated material.

The physical appearance of sample before and after cold test as well as dilution stability test is similar and there is no separation of any solid materials, indicating that physical characteristics of formulation does not produce any significant changes. Hence, it is concluded that carrabiitol formulation is stable at cold temperature.

### 13. Phytotoxicity Assessment of Carrabiitol Formulation Using Legumes Plant

Seeds of Ground nut (Arachis hypogaea) were collected from authenticated agriculture seeds shop in Ankleshwar, Gujarat. The seeds were surface sterilized with 70% IPA for 1-2 min followed by 0.3% HgCl₂ followed by sterile DW and soaked for overnight in sterile DW. A complete randomized block experimental design was used with two experimental concentrations (replications) including range of 3000 mg/L 10000 mg/L and 50000 mg/L. Selected concentration of carrabiitol was prepared by adding respective weight of formulation in MS media. Sterilized by autoclaving it at 121° C. for 20 min at 15 PSI. After seeds germination, seeds were transferred to tubes and put at 25° C. +/- 2° C. with 400 micro-mol/cm light intensity (16/8 day/night ratio), relative humidity of 65-70%.

Phytotoxicity is the capacity of a compound (such as a plant protection product) to cause temporary or long-lasting damage to plants. The following parameters were consider for phytotoxic assessment at morphological levels.
i. Modifications in the development cycle: Under this heading can be considered any inhibition or delay in emergence or growth, and all phenological modifications, particularly delays in flowering, fruiting and ripening, etc., or non-appearance of certain organs (leaves, flowers, fruits, etc.).
ii. Thinning: Loss of whole plants, by failure to emerge or to grow after transplanting, or by disappearance of plants after emergence.
iii. Modifications in colour (plant tissue not destroyed): The whole plant or parts of it may be discoloured: chlorosis, whitening, change in intensity (lighter or darker), browning, reddening. The discolouration may be localized (spots, internal or external).
iv. Necrosis: Necrosis is the local death of tissues or organs, generally appearing first as a discolouration. Necrotic spots on leaves may eventually disappear, leaving perforations.
v. Deformations: This term covers any morphological modification of the plant or part of it (including roots) making it deviate from the norm. This includes curling, rolling, stunting or elongation, change in size or volume (the latter sometimes being rated in terms of vigour). Effects such as wilting may also be considered under this heading

The morphological parameters of ground nut after 12 days presented in table 16 and dry weight of root and shoot is presented in table 17.

TABLE 16

Morphological observation of Ground nut seeds after 12 days

| Sr no | Carrabiitol Concentration | Modification in development cycle | Thinning (Failure to emerge) | Necrosis | Color Modification | Deformation |
|---|---|---|---|---|---|---|
| 1 | Control | No | No | No | No | No |
| 2 | 0.3% | No | No | No | No | No |
| 3 | 1% | No | No | No | No | No |
| 4 | 5% | No | No | No | No | No |

TABLE 17

Dry Weight of root and shoot of ground nut after 12 days

| Sr no | Carrabiitol Concentration | Root weight in g (Average of 5 replicates) | Shoot weight in g (Average of 5 replicates) |
|---|---|---|---|
| 1 | Control | 0.0778 | 0.2074 |
| 2 | 0.3% | 0.0878 | 0.2976 |
| 3 | 1 % | 0.0820 | 0.2422 |
| 4 | 5 % | 0.0888 | 0.1979 |

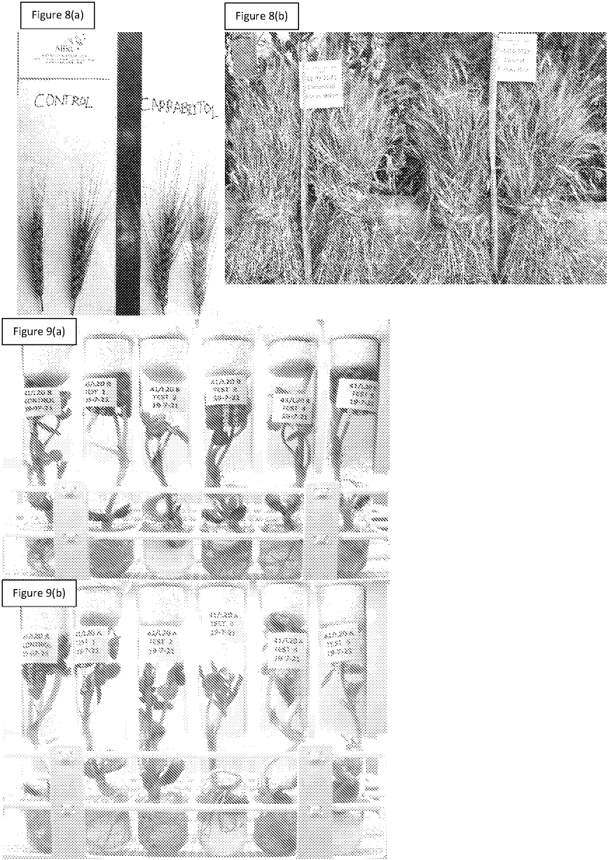

The above data represented that carrabiitol does not produce any phytotoxic effect on ground nut plants upto 5% i.e. 50000 mg/L. The pictures of 1% and 5% shown in FIG. 9.

We claim:

1. A formulation for maintaining osmotic balance in plants against abiotic stress, the formulation comprising at least one carrageenan oligosaccharide derivative polyol, wherein the carrageenan oligosaccharide derivative polyol is selected from a group of di-saccharide polyols, tri-saccharide polyols, tetra-saccharides polyols, hexa-saccharide polyols and possible derivatives at most up to about 60%, expressed in percent relative to a total carrageenan oligosaccharide derivative polyol; and at least one adjuvant ranging from 0.1% to 10%;
wherein the formulation obtained is free of bio-actives and has heavy metals not more than 10 mg/kg;
wherein the said formulation is effective at minimal dosage of 150 ml/acre;
wherein at least one carrageenan oligosaccharide derivative polyol has molecular weight in the range of 200 to 12,000 dalton;
wherein the formulation is clear and transparent, non-crystalline and stable at temperatures in the range of 1° C. to 60° C.; and
wherein the formulation has shelf life of at least three years.

2. The formulation for maintaining osmotic balance in plants against abiotic stress as claimed in claim 1, wherein the carrageenan oligosaccharide derivative polyol is obtained from red seaweed selected from Kappaphycus, Chondrus, Mastocarpus, Halymenia, Porphyra, Solieria, and combinations thereof; more preferably Kappaphycus spp.

3. The formulation for maintaining osmotic balance in plants against abiotic stress as claimed in claim 1, wherein the formulation is used for microencapsulation of microbes, and for encapsulation of fertilizers, minerals, agricultural supplements.

4. The formulation for maintaining osmotic balance in plants against abiotic stress as claimed in claim 1, wherein the formulation is effective for any type of plant species when applied to plant, planting material and plant growth supporting substrate.

5. The formulation for maintaining osmotic balance in plants against abiotic stress as claimed in claim 1, wherein the said formulation is water soluble, biodegradable, and supports growth of microorganisms.

6. The formulation for maintaining osmotic balance in plants against abiotic stress as claimed in claim 1, wherein said formulation is converted into the suitable forms selected from sprays, emulsions, suspensions, dusts, powders, pastes and granules, by employing any known mechanical and industrial methods.

7. The formulation for maintaining osmotic balance in plants against abiotic stress as claimed in claim 1, wherein the said abiotic stress is hydric stress, drought, osmotic stress, thermal stress, light stress, nutrient deficiency, and chemical stress generated by metallic or organic pollutant in soil to grow said plant.

8. The formulation for maintaining osmotic balance in plants against abiotic stress as claimed in claim 1, wherein the formulation promotes plant resistance to abiotic stress is more specifically drought, salinity, extreme temperatures and water stress among others.

9. The formulation for maintaining osmotic balance in plants against abiotic stress as claimed in claim 1, wherein said formulation is used in sustaining plant growth in abiotic stress condition, said plant growth being measured in terms of at least one of: an increased yield, increased seed germination, increased resistant to disease, early maturation, increased leaf area, stem height, leaf width, nitrogen assimilation, protein stabilization, cell division and combinations thereof.

10. The formulation for maintaining osmotic balance in plants against abiotic stress as claimed in claim 1, wherein the said formulation is stable in a wide range of pH 2 to 12 making it amiable for mixing with different agri-inputs for production of combination products and/or for field application in combination with other inputs.

11. A method for obtaining formulation comprising at least one carrageenan oligosaccharide derivative polyol from seaweed(s) as claimed in claim 1, wherein the method comprises:
a) processing the seaweed(s) to obtain first residue and first filtrate;
a) diluting the first residue obtained from step (a) with water upto 35% and adjusting pH in the range of 1 to 5.5 with organic acid and heating at a temperature in the range of 50 to 150° C. for a time period in the range of 30 minutes to 300 minutes to obtain a solution;
b) cooling the solution to ambient temperature and centrifuging/filtering to obtain second residue and second filtrate;
c) treating the second filtrate with a metallic ion complex at a temperature in the range of 30° C. to 110° C. for the time period in the range of 30 minute to 300 minutes under continuous stirring followed by neutralising with an alkali solution to obtain a neutralised solution;
d) adding a suitable preservative to the neutralised solution to obtain the carrageenan oligosaccharide derivative polyol; wherein the carrageenan oligosaccharide derivative polyol_thus obtained has_osmo-protectant activity; and
e) evaporating, drying and sieving the carrageenan oligosaccharide derivative polyol of step (e) to obtain the formulation.

12. The method for obtaining formulation comprising at least one carrageenan oligosaccharide derivative polyol from seaweed as claimed in claim 11, wherein the formulation have pH in the range of 4.5 to 7.9.

13. The method for obtaining formulation comprising at least one carrageenan oligosaccharide derivative polyol from seaweed as claimed in claim 11, wherein the carrageenan oligosaccharide derivative polyol is mixed with a specific amount of minerals, beneficial/suitable nutrients for plant growth and/or optionally binding agent to obtain granule.

14. The method for obtaining formulation comprising at least one carrageenan oligosaccharide derivative polyol from seaweed as claimed in claim 11, wherein the organic acid used in the said method is selected from a group consisting of citric acid, malic acid, ascorbic acid, lactic acid, tartaric acid, oxalic acid, maleic acid, succinic acid, acetic acid, trifluoracetic acid, phosphoric acid, phthalic acid & propionic acid and combinations thereof; preferably citric acid, malic acid or lactic acid.

15. The method for obtaining formulation comprising at least one carrageenan oligosaccharide derivative polyol from seaweed as claimed in claim 11, wherein the metallic ion complex is selected from a group consisting of sodium borohydride, potassium borohydride (KBH4), potassium iodide (KI), tin chloride, dithionates, thiosulfate, diborane and combinations thereof; preferably potassium borohydride (KBH4) or potassium iodide (KI) or combinations of both in the range of 0.01% to 10%.

16. The method for obtaining formulation comprising at least one carrageenan oligosaccharide derivative polyol from seaweed as claimed in claim 11, wherein the suitable preservative used in the said method is selected from a group consisting of sodium paraben, methyl paraben, formaldehyde, methanol, potassium sorbate, sodium benzoate and combinations thereof; more preferably formaldehyde or methanol in a range of 0.01% to 1%.

17. The method for obtaining formulation comprising at least one carrageenan oligosaccharide derivative polyol from seaweed as claimed in claim 11, wherein the said method is a zero discharge method that provides optimum yield, improves extraction efficiency, less energy consumption and maximum utilization of seaweed biomass as well as economical and viable process.

18-23. (canceled)
